(19) United States
(12) Patent Application Publication
(10) Pub. No.: US 2026/0364212 A1
(43) Pub. Date: Nov. 19, 2026

(54) INTRACELLULAR DELIVERY AND MITOCHONDRIAL TARGETING BY FLUORINATION

(71) Applicant: The Texas A&M University System, College Station, TX (US)

(72) Inventors: Lin Zhu, Kingsville, TX (US); Yin Liu, Kingsville, TX (US); Ying Tu, Kingsville, TX (US)

(73) Assignee: The Texas A&M University System, College Station, TX (US)

(21) Appl. No.: 19/577,312

(22) Filed: Mar. 24, 2026

Related U.S. Application Data
(63) Continuation of application No. 17/437,417, filed on Mar. 27, 2020, now abandoned.
(60) Provisional application No. 62/824,791, filed on Mar. 27, 2019.

(51) Int. Cl. A61K 49/00; A61K 47/69; C08G 18/28; C08G 18/71; C08G 65/337

(52) U.S. Cl. CPC A61K 49/0082; A61K 47/6907; A61K 47/6935; A61K 49/0043; A61K 49/0054; A61K 49/0093; C08G 18/283; C08G 18/2885; C08G 18/711; C08G 65/337

(57) ABSTRACT

In an embodiment, the present disclosure relates to a targeting platform that includes a targeted delivery system including an agent. In some embodiments, the targeted delivery system is modified by fluorination. In some embodiments, the targeted delivery system is electrically charge neutral. In a further embodiment, the present disclosure relates to a method that includes fluorinating a targeting compound with a fluorinating reagent and loading an agent with the fluorinated targeting compound to thereby form a micelle. In an additional embodiment, the present disclosure relates to a targeting platform that includes a fluorinated polymeric system including an agent. In another embodiment, the present disclosure relates to a method that includes fluorinating a targeting compound with a fluorinating reagent and loading an agent with the fluorinated targeting compound to thereby form a micelle. Additionally, the method can include administering the fluorinated targeting compound to a subject.

### CROSS-REFERENCE(S) TO RELATED APPLICATIONS

This patent application is a continuation of U.S. application Ser. No. 17/437,417 filed Sep. 9, 2021, which is a U.S. National Stage Application of PCT/US2020/025382 filed Mar. 27, 2020, which claims priority from, and incorporates by reference the entire disclosure of U.S. Provisional Application No. 62/824,791 filed on Mar. 27, 2019.

### BACKGROUND

This section provides background information to facilitate a better understanding of the various aspects of the disclosure. It should be understood that the statements in this section of this document are to be read in this light, and not as admissions of prior art.

Mitochondria play a role in various physiological and pathological processes and mitochondrial targeting is desired for many therapeutics. In this disclosure, a self-assembling fluoroamphiphile is synthesized and evaluated as a micellar nanocarrier for intracellular delivery and mitochondrial targeting. The fluorinated micelles can efficiently enter various cells via clathrin-mediated endocytosis, rapidly escape from endosomes via membrane fusion, and preferentially accumulate in mitochondria rather than any other intracellular compartments. Unlike popularly used mitochondria-targeted lipophilic cations, these fluoroamphiphiles are electrically neutral. Their mitochondrial targeting is potential-independent and also does not alter the mitochondrial membrane potential and mitochondrial health. The present disclosure represents a reliable intracellular delivery strategy allowing for efficiently and specifically targeting of imaging and therapeutic agents to mitochondria and also provides a better understanding of subcellular localization of fluorinated materials.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure relates to a targeting platform that includes a targeted delivery system including an agent. In some embodiments, the targeted delivery system is modified by fluorination. In some embodiments, the targeted delivery system is electrically charge neutral. In some embodiments, the targeted delivery system forms a micelle around the agent.

In a further embodiment, the present disclosure relates to a method that includes fluorinating a targeting compound with a fluorinating reagent and loading an agent with the fluorinated targeting compound to thereby form a micelle. In some embodiments, the fluorinated targeting compound surrounds the agent.

In an additional embodiment, the present disclosure relates to a targeting platform that includes a fluorinated polymeric system including an agent. In some embodiments, the polymeric system is derived from polyethylene glycol. In some embodiments, the fluorinated polymeric system is electrically charge neutral. In some embodiments, the fluorinated polymeric system forms a micelle around the agent.

In another embodiment, the present disclosure relates to a method that includes fluorinating a targeting compound with a fluorinating reagent and loading an agent with the fluorinated targeting compound to thereby form a micelle. In some embodiments, the fluorinated targeting compound surrounds the agent. In some embodiments, the method further includes administering the fluorinated targeting compound to a subject, targeting, by the fluorinated targeting compound, an organelle in the subject, and delivering the agent to the organelle.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates synthesis of the PEG-F7 fluoroamphiphile (mPEG-MAL, methoxy polyethylene glycol (1k, 2k, or 5k Da) maleimide);

FIG. 2A shows a schematic illustration of the PEG-F7 and its assembled micelle;

FIG. 2B illustrates endocytosis, endosomal escape, and mitochondrial targeting using the fluoroamphiphile-assembled polymeric micelles;

FIG. 3 illustrates cellular uptake of the rhodamine B-phosphatidylethanolamine (Rh-PE)-loaded polymeric micelles in Hela cells, determined by flow cytometry (n=3). The PEG2k-PE micelles were modified by PEG2k-F7 (F7) or TAT-PEG2k-PE (TAT) at indicated ratios at an incubation time of 1 hour;

FIG. 4A, FIG. 4B and FIG. 4C illustrate the effect of endocytosis inhibitors on cellular uptake, as determined by flow cytometry. The Hela cells were pre-incubated with the endocytosis inhibitors, chlorpromazine (FIG. 4A) nystatin (FIGS. 4B) and 2-hydroxypropyl-P-cyclodextrin (HP-P-CD) (FIG. 4C), for 30 min, and then incubated with the rhodamine B-phosphatidylethanolamine (Rh-PE)-loaded micelles for additional 60 min (n=3);

FIG. 5A and FIG. 5B illustrates the effect of PEG2k-F7 on the lipid membrane fusion at different pHs (pH 5.0 and pH 7.4) determined by fluorescence resonance energy transfer (FRET) and shows a schematic illustration of the lipid bilayer fusion experiment;

FIG. 6A and FIG. 6B illustrates the potential-independent mitochondrial targeting of PEG2k-F7; FIG. 6A illustrates subcellular localization of the Rh-PE-loaded micelles with the mitochondria (stained by MitoView™ Green), endoplasmic reticulum (stained by the ER-Tracker™ Green), and Golgi apparatuses (stained by the NBD C6-ceramide) in Hela cells. Cell nuclei were stained by Hoechst 33258 (blue); FIG. 6B illustrates the mitochondrial potential upon polymer incubation. The mitochondrial membrane potential was measured by the potential-sensitive JC-1 dye that forms red aggregates in healthy, “negatively charged” mitochondria and becomes green monomers in the cytoplasm when the mitochondria undergo depolarization.

FIG. 7 illustrates fluorine-containing reagents according to some embodiments of the present disclosure;

FIG. 8A and FIG. 8B illustrate biodistribution of Rh-PE (dye), control nanoparticles, and nanoparticles of the presented disclosure in 4Tl breast cancer-bearing mice;

FIG. 9 illustrates in vivo accumulation in tumoral mitochondria measured by flow cytometry for untreated, Rh-PE (dye), control nanoparticles, and nanoparticles of the present disclosure cells over 2 h, 8 h, and 24 h;

FIG. 10 illustrates body weight of mice treated with saline, empty nanoparticles of the present disclosure, Vitamin E Succinate (VES; drug), control nanoparticles, and nanoparticles of the present disclosure over 14 days;

FIG. 11 illustrates tumor size reduction of mice treated with saline, empty nanoparticles of the present disclosure, VES (drug), control nanoparticles, and nanoparticles of the present disclosure over 14 days;

FIG. 12 illustrates tumor weight of mice treated with saline, empty nanoparticles of the present disclosure, VES (drug), control nanoparticles, and nanoparticles of the present disclosure; and

FIG. 13 illustrates white blood cell counts of mice treated with saline, empty nanoparticles of the present disclosure, VES (drug), control nanoparticles, and nanoparticles of the present disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

The fluorination of polymers or nanoparticles is able to enhance intracellular delivery and mitochondrial targeting. Unlike currently used mitochondria-targeted lipophilic cations, these fluorinated polymers are electrically neutral. Their self-assembled micelles enter cells efficiently via endocytosis, and then rapidly escape from endosomes. After internalization, the micelles and their loaded cargoes, including drugs and fluorescent dyes, are preferentially accumulated in mitochondria in a potential-independent manner, resulting in mitochondrial targeting and enhanced drug efficacy. In some embodiments, the present disclosure provides a novel intracellular delivery strategy allowing for efficiently and specifically targeting of imaging and therapeutic agents to mitochondria.

The data, discussed in further detail herein, illustrate that the fluorine-containing materials, including fluorinated polymers, for example, had enhanced cellular uptake and mitochondria accumulation. The conjugation of fluorine (fluorination) presented herein is a novel way for intracellular delivery of drugs and imaging agents to mitochondria. Unlike the popularly used lipophilic cations, fluorination will not change the charges of the materials, which will minimize the charge-induced nonspecific interaction and facilitate drug delivery and targeting.

In some embodiments, fluorination increases the efficiency of intracellular delivery and mitochondrial targeting. In various embodiments, intracellular delivery of drugs, such as, but not limited to, molecular probes and nanoparticles, to mitochondria via a charge independent manner are enhanced by fluorination. In some embodiments, the fluorinated amphiphilic polymers are readily self-assembled to a micellar nanocarrier for delivery of hydrophobic molecules. In some embodiments, the self-assembled micelles enter cells efficiently via clathrin-mediated endocytosis, and then rapidly escape from endosomes. In some embodiments, various mitochondria-targeted drugs and imaging probes can be easily prepared, which provide higher biocompatibility due to the neutral charge. The currently used mitochondria-targeted therapeutics and dyes are based on the lipophilic cations. The positive charge may cause nonspecific interaction with negatively charged biological molecules and cells. In some embodiments, the mitochondria-targeted therapeutics efficiently targets intracellular mitochondria without changing the charge of drugs or dyes.

Mitochondria, a key organelle, are considered the energy generators and metabolic signaling centers, supporting a wide range of physiological and pathological processes of mammalian cells. Mitochondrial health is important and their defects or dysfunctions have been connected with many human diseases and disorders, such as neurological diseases, cardiovascular diseases, cancer, and the like. Mitochondria have become one of the most important therapeutic targets and specific delivery of therapeutics or imaging probes to mitochondria is highly desired for studying mitochondrial functions and treating mitochondria-related diseases.

However, because of the hydrophobicity, dense double membrane, and negative potential, mitochondria are difficult to access by many therapeutics. Over the past decade, significant effort has been made to develop mitochondria-targeted systems for delivery of drugs and imaging agents. Among them, various mitochondria-targeted lipophilic cations, including triphenylphosphonium (TPP)-based cations, rhodamine, cyanine cations, and cationic peptides, have been developed to modify drug molecules or their carriers for the enhanced binding affinity with negatively charged mitochondria. Although promising, the positive charge is not always favorable to biomedical applications, particularly drug delivery, and may cause nonspecific drug biodistribution and undesirable side effects. On the other hand, the potential-driven mitochondrial targeting would be compromised if the mitochondria undergo depolarization during cell apoptosis or drug treatments.

Due to the unique properties, fluorine has been exploited extensively in drug discovery and development to enhance the therapeutic activity and increase chemical or metabolic stability of numerous drugs. Fluorination is also an effective technique to improve the stability and specificity of proteins and prepare molecular probes for magnetic resonance imaging and positron emission tomography imaging.

In some embodiments, the compositions of the present disclosure can be fluorinated by a fluorinating agent such as, for example, fluorine containing compounds, heptafluorobutyric acid, nonafluorovaleric acid, pentafluoropropionic acid, heptafluorobutylamine, or combinations thereof. In some embodiments, the compositions of the present disclosure can be small molecules, macromolecules, nanoparticles, or combinations thereof. For example, in some embodiments, the compositions of the present disclosure can be FITC or PEG. In some embodiments, the compositions of the present disclosure can function as mitochondrial targeting, mitochondrial acting, therapeutics, diagnostics, imaging probe, imaging dye, or combinations thereof.

As discussed in further detail below, the present disclosure demonstrates that the enhanced intracellular delivery and mitochondrial targeting can be achieved using the fluorinated polymers or their assembled nanoparticles.

Reference will now be made to more specific embodiments of the present disclosure and data that provides support for such embodiments. However, it should be noted that the disclosure below is for illustrative purposes only and is not intended to limit the scope of the claimed subject matter in any way.

Materials. Methoxy polyethylene glycol maleimide (PEG-MAL, 1k, 2k and 5k Da), 1,2-distearoyl-sn-glycero-3-phosphoethanolamine N-[amino (polyethylene glycol)] (PEG-PE, 1k, 2k and 5k Da), and maleimide-polyethylene glycol (2k Da)-succinimidyl valerate (MAL-PEG2k-SV A) were purchased from Laysan Bio, Inc. (Arab, AL, USA). Heptafluorobutyric acid (F7-COOH) was purchased from Matrix Scientific Co. (Columbia, SC, USA). 1H, 1H-Heptafluorobutylamine (F7-NH2) was purchased from TCI America (Boston, MA, USA). 1,2-Dioleoyl-sn-glycero-3-phosphoethanolamine (DOPE), 1,2-dipalmitoyl-sn-glycero-3-phosphocholine (DPPC), and 1,2-dioleoyl-sn-glycero-3-phosphoethanolamine-N-(lissamine rhodamine B sulfonyl) (Rh-PE) were purchased from Avanti Polar Lipids, Inc. (Alabaster, AL, USA). TLC plates (silica gel 60 F254) were purchased from EMD Biosciences (La Jolla, CA, USA). N-hydroxysuccinimide (NHS), 2′,7′-dichlorodihydrofluorescein diacetate (H2DCFDA), chlorpromazine hydrochloride, nystatin, pyrene, and 1-(3-dimethylaminopropyl)-3-ethylcarbodiimide hydrochloride (EDC-HCl) were purchased from Sigma-Aldrich Chemicals (St. Louis, MO, USA). L-cysteine ethyl ester hydrochloride, triethylamine (TEA), and 2-hydroxypropyl-P-cyclodextrin (HP-P-CD) were obtained from Alfa Aesar (Haverhill, MA, USA). 3.3′-dioctadecyloxacarbocyanine perchlorate (DiO), 1,1′-dioctadecyl-3,3,3′,3′-tetramethylin docarbocyanine perchlorate (Dil), fluorescein isothiocyanate (FITC), rhodamine 123, chloroform, dichloromethane (DCM), dimethylformamide (DMF), methanol, ethyl acetate, hexane, ER-TRACKER™ Green, and Mitochondria Isolation Kit for Cultured Cells were purchased from Thermo Fisher Scientific (Rockford, IL, USA). d-Vitamin E succinate (VES) was purchased from Spectrum Chemical Manufacturing Corp. (New Brunswick, NJ, USA). MITOVIEW™ Green, MITOVIEW™ 633, NBD C6-ceramide, and JC-1 mitochondrial membrane potential detection assay kit were purchased from Biotium (Fremont, CA, USA), LYSO-ID® green detection kit and Hoechst 33258 were purchased from Enzo Life Science (Farmingdale, NY, USA).

Cell Lines. The human ovarian cancer cells (NCVADR-RES), human cervical cancer cells (HeLa), and mouse fibroblast cells (NIH/3T3) were grown in complete growth media (DMEM supplemented with 100 U-mL−¹ penicillin, 100 μg-mL−¹ streptomycin, and 10% PBS) at 37 °C in 5% CO2.

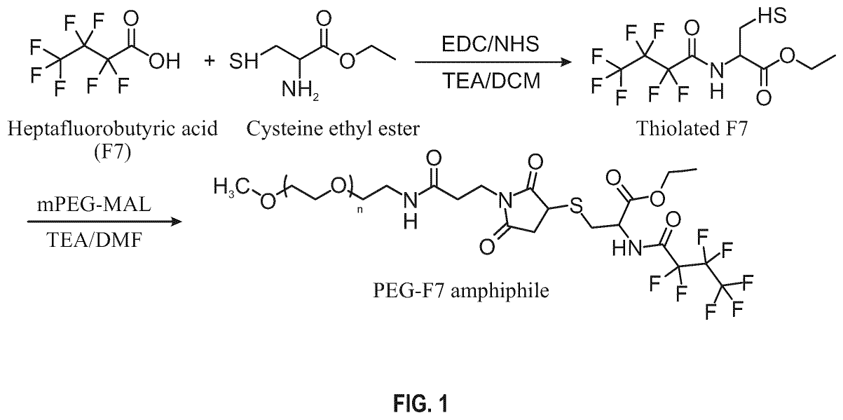

Synthesis, Purification, and Characterization of PEG-F7 Copolymers. The similar synthesis, purification, and characterization protocols were used for all of the PEG-F7 copolymers (PEG1k-F7, PEG2k-F7, and PEG5k-F7) (FIG. 1). Here, the synthesis of PEG2k-F7 was used as an example. First, the F7-COOH (2.5 mmol) was activated by the excess amount of the coupling reagents (EDC/HOBT) and reacted with L-cysteine ethyl ester hydrochloride (3 mmol) in the DCM in the presence of a trace amount of triethylamine at room temperature overnight. The F7-SH was purified by the column chromatography (ethylacetate/hexane, 1:1, v/v), affording 455 mg of F7-SH (−48% yield) as a white powder. Then, the F7-SH was reacted with the PEG derivative, PEG2k-MAL, at a molar ratio of 1.2:1 in the DMF in the presence of triethylamine at room temperature overnight. The crude product was purified by the dialysis (MWCO 1000 Da) against water for 24 h, followed by the freeze drying, affording the PEG2k-F7(−75% yield) as a white powder.

The product was characterized by thin layer chromatography (TLC) in the solvent mixture of methanol and chloroform (1:4, v/v) and the TLC plate was stained to visualize the PEG chain by the Dragendorff s reagent. The polymer's chemical structure and micelle formation were analyzed by H NMR spectroscopy using the deuterated solvents, DMSO-d6, CD3OD-d4, and D2O-d2, respectively. For the matrix assisted laser desorption ionization time of flight mass spectrometry (MALDI-TOF MS), the trans-2-[3-(4-tert-Butylphenyl)-2-methyl-2-propenylidene] malononitrile (DCTB) was used as a matrix. The polymer/DCTB mixture was deposited on a stainless steel sample holder and analyzed on a Bruker Daltonics Microflex mass spectrometer (Billerica, MA) in positive deflectron mode.

Determination of Critical Micelle Concentration. The critical micelle concentration (CMC) was determined using pyrene as a hydrophobic fluorescent probe. Briefly, the pyrene chloroform solution was added to the testing tube at the final concentration of 8×10−⁵ Mand the polymers in chloroform were added to the tubes at a 10-fold serial dilution (from 10° to 10-s mg-mL−¹). The tube was dried overnight under vacuum, and then was hydrated by PBS and incubated with shaking at room temperature for 24 h before measurement. The fluorescence intensity was measured on a Tecan Infinite MIO00 Pro microplate reader at λₑₓ 338 nm and 334 nm and λₑₘ 390 nm. The intensity ratio (1338 nm/I 334 nm) was calculated and plotted against the polymer concentration. The CMC value was obtained as the crossover point of the two tangents of the curves.

Particle Size and Zeta Potential Measurement. The polymers were hydrated at various pHs (5.0, 7.4, and 8.5) and methanol at a final concentration of 0.1 mg-mL−¹. The particle size of the self-assembled micelles was measured by dynamic light scattering (DLS) on a NanoBrook 90Plus PALS Zeta Potential Analyzer (Brookhaven Instruments) at 25 °C. The zeta potential of the micelles in aqueous buffers was measured by the same instrument.

Fluorescence Resonance Energy Transfer (FRET). The micelle stability and the lipid fusion were studied by the FRET. For the micelle stability study, one milliliter of the polymeric micelles containing 100 μg polymers, 1 μg DiI, and 1 μg DiO were incubated in the PBS or mouse serum at 37 °C over 24 h. Their time-resolved spectra were recorded by the microplate reader at Nex 484 nm and λₑₘ from 490 nm to 700 nm. To study the lipid membrane fusion, the polymers (0.1 mg/mL) were incubated with the DPPC (2 mg/mL) liposomes (half of them containing Dil/DiO) at pH 5.0 or 7.4 at 37 °C. The time-resolved spectra were recorded for 1 h at λₑₓ 484 nm and λₑₘ from 490 nm to 700 nm.

Cellular Uptake. The rhodamine B-phosphatidylethanolamine (Rh-PE) (1%, w/w) was used as a fluorescent indicator to prepare the micelles for the cellular uptake study. Before the experiment, the HeLa, NCI/ADR-RES, or NIH/ 3 T3 cells were seeded at a density of 1×10⁵ cells/well in 24-well plates for 24 h. After washing with PBS, the cells were incubated with the Rh-PE loaded micelles (polymer concentration: 0.1mg-mL−¹ ) in a serum-free medium for 60 min. Then, the cells were washed three times by PBS to remove the uninternalized micelles/dyes. For the microscopy, the cells were observed on a Nikon Ti Eclipse fluorescence microscope system. For the flow cytometry, the cells were trypsinized and harvested by centrifugation (600 g for 4 min). The collected cells were washed with PBS and resuspended in 400 μL PBS, followed by the analysis on a BD Accuri C6 flow cytometer. The dead cells and cell debris were excluded from viable cells using the forward scatter and side scatter.

To compare with the TAT-modified micelles (TAT-PEG2k-PE/PEG2k-PE), the PEG2k-F7 containing micelles (PEG2k-F7/PEG2k-PE) (at the molar ratio of 0:100, 10:90, and 100:0) were prepared. The Rh-PE was loaded to the micelles and their cellular uptake was analyzed by the flow cytometry.

To study the influence of energy/temperature on cellular uptake, the cells were pre-incubated at 4 °C for 60 min, followed by incubation with the Rh-PE-loaded micelles for additional 60 min at 4 °C. The cellular uptake was analyzed by the flow cytometry.

To study the internalization mechanisms, the cells were pre-incubated with the endocytosis inhibitors, chlorpromazine hydrochloride, nystatin, or HP-P-CD for 30 min, followed by incubation with the Rh-PE-loaded micelles for additional 60 min. The cellular uptake was analyzed by the flow cytometry.

Subcellular Localization and Mitochondrial Targeting. The HeLa, NCVADR-RES, or NIH/3T3 cells were seeded at a density of 1×10⁵ cells/well on a 15 mm glass bottom cell culture dish for 24 h. The cells were incubated with the Rh-PE-loaded micelles (polymer concentration: 0.1 mg·mL−¹) in the serum-free medium at 37 °C. The intracellular compartments were stained by the following commercial dyes using the vendors' recommended protocols. Then, the cells were analyzed on a Nikon Ti Eclipse confocal microscope system.

To study the endocytosis and endosomal escape, the living cells were stained by the LYSO-ID® Green for 30 min to visualize the endosomes/lysosomes and also stained by Hoechst 33258 (2 M) for 15 min to visualize the cell nuclei.

To study the intracellular localization of the PEG2k-F7 micelles, mitochondria were stained by Rh 123 (25 nM) or MITOVIEW™ Green (100 nM) for 30 min, endoplasmic reticulum was stained by ER-TRACKER™ Green (1 μM) for 30 min, and Golgi apparatuses were stained by NBD C6-ceramide (5 M) for 30 min. To visualize cell nuclei, the cells were fixed by 4% paraformaldehyde for 5 min at room temperature, and incubated with 2 μM Hoechst 33258 for 1 min.

To study the underlying mechanisms of mitochondrial targeting, the PEG2k, F7, and PE were labeled by the fluorescein isothiocyanate (FITC). The samples (FITC-PEG2k, FITC-PE, FITC-F7, PEG2k-PE/FITC-F7, or Rh123) were pre-normalized to the same fluorescence intensity and then incubated with the Hela cells at 37 °C for 1 h. The mitochondria were stained by the MITOVIEW™ 633 for 30 min. Then, the cells were fixed by 4% paraformaldehyde for 5 min at room temperature, and incubated with 2 μM Hoechst 33258 for 1 min.

Mitochondrial Membrane Potential Measurement. The mitochondrial membrane potential was determined using the JC-1 dye by flow cytometry. Briefly, the Hela cells were seeded at a density of 1×10⁵ cells/well in 24-well plates for 24 h. The cells were incubated with the same molar concentration (50 μM) of the PEG2k-F7, TPP and CCCP (mitochondrial uncoupler) in the serum-free medium for 1 h. Then, the cells were washed three times by the iced-cold medium to remove the uninternalized materials. The cells were incubated with the JC-1 dye (Biotium) for 15 min at 37 °C, followed by the flow cytometry.

Mitochondria Isolation and Binding Affinity Study. The intracellular mitochondria were isolated by the commercial Mitochondria Isolation Kit for Cultured Cells (Thermo Fisher Scientific). Briefly, 2×10⁷ Hela cells were collected by trypsinization and centrifugation (850 g for 2 min). Then, the cells were lysed and the mitochondria were separated from other cell fragments by several washing and centrifugation steps as the manufacture's manual (Option A). The isolated mitochondria were freshly made for the binding affinity study.

The isolated mitochondria were incubated with the FITC-PEG2k, FITC-PE, FITC-F7, FITC-PEG2k-F7, Rh 123, and MITOVIEW™ Green at 37 °C for 1 h, respectively. Then, the mitochondria were washed with PBS for three times and suspended in 200 μL PBS. The mitochondria suspension was observed on a Nikon Ti Eclipse fluorescence microscope. The fluorescence intensity was recorded on the microplate reader.

For the competitive binding assay, the isolated mitochondria were pre-incubated with triphenylphosphonium (TPP) or PEG2k-F7 at 37 °C for 1 h, and then washed with PBS for three times. The pretreated mitochondria were incubated with Rh 123 at 37 °C for additional 1h. After washing with PBS for three times, the mitochondria were analyzed by the microplate reader.

For quantification of the mitochondria-accumulated dyes in living cells, 2×10⁷ cells were incubated with the FITC-PE, FITC-F7, or Rh123 at 37 °C for 1 h. After washing with PBS, the (total) cellular fluorescence intensity was measured by microplate reader. Then, the cells were lysed and the mitochondria were separated from other cell fragments by Mitochondria Isolation Kit for Cultured Cells (Option A). The fluorescence intensities of the isolated mitochondria and other cell fragments were measured by the microplate reader. To study the influence of mitochondrial membrane potential on mitochondrial targeting, the cells were pre-incubated with the uncoupler, CCCP (50 μM), for 15 min, and then incubated with the FITC-F7 or Rh123 for 1 h.

Intracellular Reactive Oxygen Species (ROS) Determination. The ROS production was determined using 2′,7′-dichlorodihydrofluorescein diacetate (H2DCFDA). Briefly, the Hela cells were seeded in 12-well plates at 1.0×10⁵ cells per well and incubated at 37 °C overnight before the experiment. The PEG2k-F7, VES, physical mixture (PEG2k-F7+VES), or YES-loaded micelles were incubated with the cells for 20 h. After washing with PBS, the treated cells were incubated with H2DCFDA (10 μM) at 37 °C for 30 min, followed by the confocal microscopy.

Cytotoxicity. The HeLa, NCVADR-RES or NIH/3T3 cells were seeded in the 96-well plates at 2×10³ cells/well 24 h before the treatment. The polymers, VES, and YES-loaded micelles (drug loading: −1%, w/w) were incubated with the cells in complete growth media, respectively at 37 °C for 48 h. The cell viability was determined by the CELLTITER-BLUE® Cell Viability Assay (Promega). Briefly, 10 μL of CELLTITER-BLUE® reagent was diluted with 90 μL of complete growth media per well and incubated with the cells at 37 °C for 2 h. Thereafter, the fluorescence intensity was recorded at λₑₓ 560 nm and λₑₘ 590 nm on the microplate reader.

Animal experiments. The subcutaneous (s.c.) syngeneic 4Tl tumor mouse model was established by s.c. implantation of 5×10⁶ 4Tl cells in the right rear flank of the BALB/c mice. The tumor was measured by a caliper.

The Rh-PE was used to substitute drug for fluorescence imaging. At 2 h, 8 h and 24 h after intravenously (i.v.) injection, mice were anesthetized and killed. The tumor, blood and major organs (heart, liver, spleen, lung, and kidney) were collected. The total fluorescence intensity in the tissues was recorded at 1 vex 560 nm and km 590 nm on a microplate reader.

To determine the in vivo cellular uptake, the fresh tissues were incubated with Collagenase D (1 mg/mL) for 30 min at 37 °C to dissociate cells. The single-cell suspension was analyzed immediate by flow cytometry.

To determine the mitochondrial accumulation, the mitochondria were isolated by the commercial Mitochondria Isolation Kit, and the mitochondrial suspension was analyzed by flow cytometry.

To study anticancer activity, the saline, YES, PEG2k-F7, YES-loaded PEG2k-PE micelles and YES-loaded PEG2k-F7 micelles were injected intravenously via the tail at a dose of 20 mg/kg every three days for 3 injections. The tumor size and mouse body weight were monitored every other day. The mice were sacrificed and the blood, major organs and tumors were collected. White blood cells were counted by using a hemocytometry by microscopy.

Statistical Analysis. Data were expressed as mean±standard deviation (SD) and analyzed by t-test using the GraphPad Prism 6.0. P<0.05 is considered to be statistically significant.

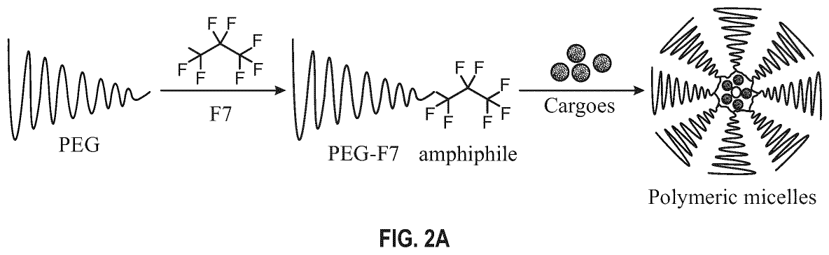

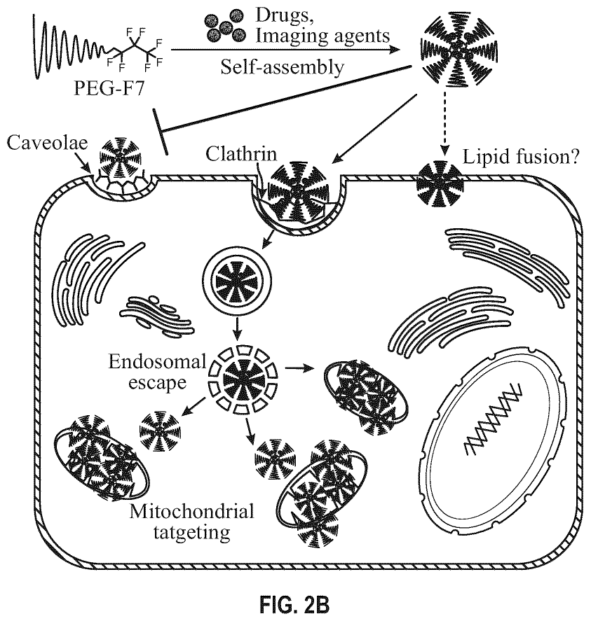

The present disclosure demonstrates that the enhanced intracellular delivery and mitochondrial targeting can be achieved using the fluorinated polymers or their assembled nanoparticles. Here, the hydrophilic polyethylene glycol (PEG) was fluorinated by heptafluorobutyric acid (F7) via a facile conjugation method, to form fluoroamphiphiles, PEG-F7 (FIG. 1 and FIG. 2A). To study the influence of the PEG on the properties of the fluoroamphiphile, three lengths of PEGs (1k, 2k, and 5k) were used. The thin layer chromatography (TLC), ¹H NMR and MALDI-TOF MS results indicated that the PEG-F7 was successfully synthesized. In the H NMR, the amide hydrogen (-NHCO) near the F7 moiety was detected when the polymer was dissolved in DMSO-d6, while its peak was disappeared when the polymer was only dispersed in D2O-d2 or methanol-d4, indicating that the PEG-F7 could self-assemble to the micellar structure due to the strong fluorocarbon-fluorocarbon interaction and PEG's hydrophilicity. The micelle formation of the fluoroamphiphiles was confirmed by their low critical micelle concentration (CMC) values (in the micromolar range) and tiny mean particle sizes (~80-150 nm). The micelles' sizes were not significantly different at various pHs and even in pure methanol, a solvent for disrupting the polymeric micelles, such as the PEG-phosphatidylethanolamine (PE)-assembled micelles. These data suggested that the PEG-F7 had excellent self-assembly capability and their assembled micelles were fairly stable. Unlike the lipophilic cation-based delivery systems, the PEG-F7-assembled micelles with the PEG shell/corona were near neutral (between −5 and +5 mV) in aqueous environments, suggesting that they would have good biocompatibility in biomedical applications, particularly drug delivery. A slight shift in zeta potential was observed when the pH changed, most likely due to the PEG-F7's pKa (−8.1±0.6, estimated by the ACD/I-Lab 2.0). The fluorescence resonance energy transfer (FRET) data showed that the dye pair-loaded PEG-F7 micelles were stable in both aqueous buffers and mouse serum at 37 °C for at least 24 h, as evidenced by their negligible dye “leakage”, ensuring the successful delivery of loaded cargoes. FIG. 2B illustrates endocytosis, endosomal escape, and mitochondrial targeting using the fluoroamphiphile-assembled polymeric micelles.

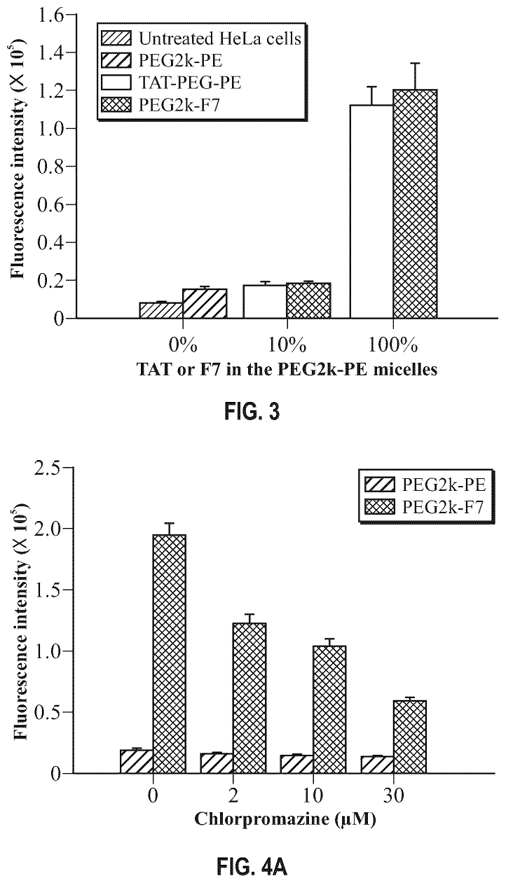

Fluorinated cationic materials, such as dendrimers and polyethylenimines, have been developed for intracellular delivery of genes and proteins. Here, to study the cellular uptake of the charge-neutral PEG-F7 micelles, the PEG-PE micelles, one of the mostly investigated micellar drug carriers, were used as a control. In addition, the rhodamine B-phosphatidylethanolamine (Rh-PE), a widely used hydrophobic dye without mitochondrial preference, was physically loaded into the micelles as a fluorescent probe. Interestingly, in spite of being electrically neutral, the PEG-F7 micelles showed excellent cellular uptake, while the PEG-PE micelles could not efficiently enter cells, implying that the F7 moiety was the major contributor to the efficient cellular uptake. High uptake was observed for all PEG (1k, 2k, and 5k)-F7 assembled micelles in both normal (NIH/3T3) and cancer cells, including drug-sensitive (HeLa) and multidrug resistant (NCVADR-RES) cells, indicating that the uptake of the PEG-F7 micelles was cell type-independent. Compared to the PEG1k or PEG5k polymers, the PEG2k-F7 had a significantly higher cellular uptake. The similar trend was observed in the PEG-PE micelles. This was probably due to the proper balance between the hydrophilicity (of PEG moiety) and lipophilicity (of F7 moiety) in the amphiphilicpolymers. Surprisingly, it was also found that the PEG2k-F7 micelles were as efficient as the PEG2k-PE micelles surface-modified with the trans-activating transcriptional activator (TAT), a well-known intracellular delivery ligand, in terms of cellular uptake (FIG. 3). Here, the amphiphilic perfluoroalkyl compounds are capable of incorporating into the phospholipid bilayer (cell membrane) with high affinity due to fluorocarbons' higher hydrophobicity, to promote cell internalization.

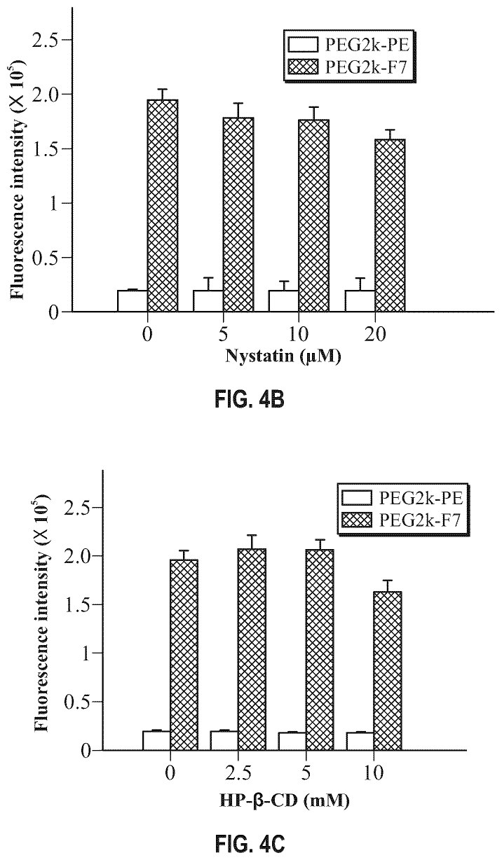

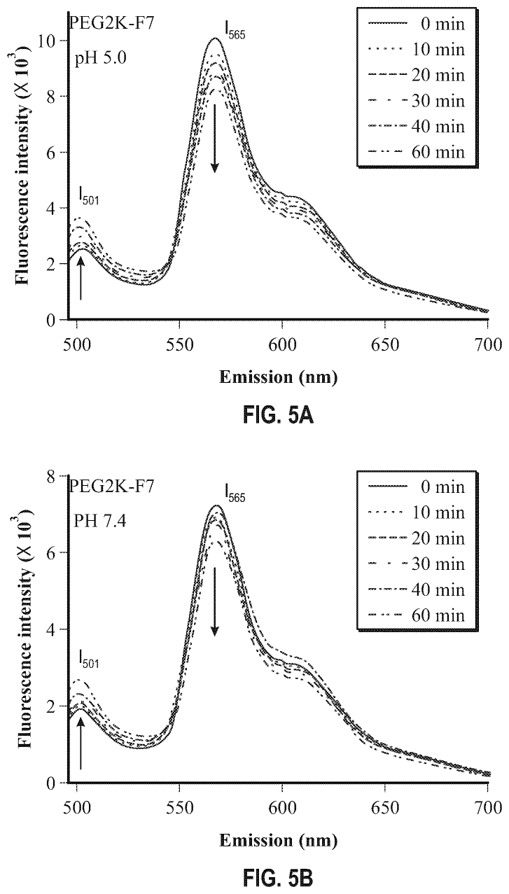

To investigate the uptake pathway, the cellular uptake was also performed at 4 °C or in the presence of endocytosis inhibitors. It was found that the uptake of the PEG2k-F7 micelles at 4 °C was only about 50% of that at 37 °C. Furthermore, the uptake of the PEG2k-F7 micelles was significantly inhibited by chlorpromazine, an inhibitor of clathrin-mediated endocytosis, rather than the inhibitors of caveolae-or lipid rafts-mediated endocytosis (nystatin and 2-hydroxypropyl-P-cyclodextrin) (FIG. 4A-FIG. 4C). These data suggested that the PEG2k-F7 micelles entered cells mainly via the energy-dependent clathrin-mediated endocytosis. A dynamic study showed that at 15 min after internalization, most of the PEG2k-F7 micelles were colocalized with the endosomes, while at 30 and 60 min, the micelles and endosomes were separated each other. The data indicated that the PEG2k-F7 micelles were efficiently internalized by cells, and then rapidly escaped from the endosomal compartment. These features would protect the loaded cargoes from endolysosomal degradation and facilitate targeting other intracellular compartments/organelles. Further studies suggested that the PEG2k-F7-induced quick lipid membrane fusion (particularly at pH 5.0) (FIG. SA) account for the efficient endosomal escape. However, the lipid fusion (at pH 7.4) (FIG. SB) induced nonendocytic internalization of the PEG2k-F7 micelles could not be ruled out, which might account for the moderate cellular uptake at 4 °C. During the uptake processes, the micelles were stable and no loaded cargoes were released, as evidenced by the color co-localization.

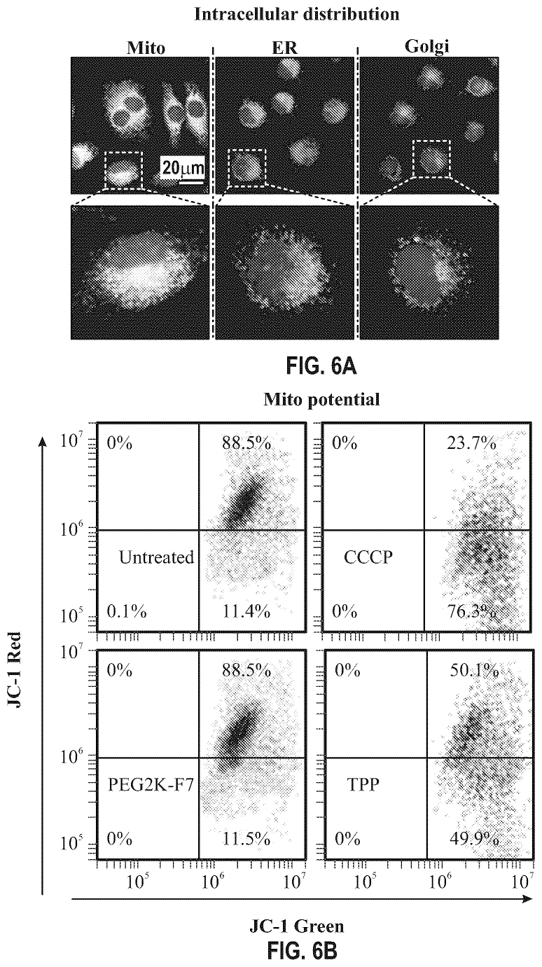

Though the fluorinated materials/nanoparticles have showed the enhanced cellular uptake, their intracellular localization pattern is unclear. Without being bound by theory, it is hypothesized that the extreme hydrophobicity of the fluorocarbon might benefit the fluoroamphiphiles' intracellular accumulation in the hydrophobic, membrane-rich organelles, such as mitochondria. To visualize the intracellular mitochondria, three commercially available mitochondrial dyes, including the MITOVIEW™ Green (potential-independent, green fluorescence), MITOVIEW™ 633 (potential-dependent, red fluorescence), and rhodamine 123 (Rh123) (potential-dependent, green fluorescence), were used. At 5 min upon incubation, the Rh-PE-loaded PEG2k-F7 micelles could be observed mainly on the cell surface, indicating a rapid onset of cellular uptake. At 30 and 60 min, the micelles were distributed to intracellular compartments and mainly co-localized with the mitochondria. In contrast, the PEG2k-PE micelles did not show significant cellular uptake even after 60 min incubation. The similar results were obtained in different cell lines and visualized by both potential, dependent and independent, mitochondrial dyes. These data were well consistent with the micelles' efficient cellular uptake and rapid endosomal escape. To further verify the PEG2k-F7 micelles' subcellular localization pattern, in addition to staining mitochondria (and nuclei), other important organelles were also stained, endoplasmic reticulum (ER) and Golgi apparatus, by ER-TRACKER™ and NBD C6-ceramide, respectively (FIG. 6A). Though these organelles also have membrane structures, the PEG2k-F7 showed low binding affinity with them. All these results strongly suggested that the PEG2k-F7 micelles could quickly enter the cells and preferentially accumulate in the mitochondria. Since the ER and mitochondria might join together at multiple contact sites, so called mitochondria-ER associated membranes, a slight ER “binding” was observed. In addition to working as a micellar nanocarrier, the PEG2k-F7 could incorporate into the liposomes and work as a mito-targeted ligand.

To study the underlying mechanisms of mitochondrial targeting, the PEG2k-F7, PEG2k, F7, and PE were labeled by fluorescein isothiocyanate (FITC), respectively, and the mitochondria were stained by MITOVIEW™ 633. Upon 1 h incubation, the FITC-PEG2k failed to enter cells due to the PEG's “stealth” property, while both FITC-PE and FITC-F7 could be internalized. After internalization, the FITC-PE was nonspecifically distributed in intracellular compartments, as evidenced by the significantly separate green and red fluorescence, indicating that the PE had no mitochondrial preference. In contrast, the FITC-F7 entered cells and then were predominantly accumulated in mitochondria, suggesting that the fluorocarbon (F7) was mainly responsible for the mitochondrial targeting. However, the F7 was not suitable as a drug carrier by itself due to its extremely high hydrophobicity. Here, the PEG imparted the hydrophilicity to the PEG-F7 and balanced the ratio of hydrophilicity to hydrophobicity, to allow the self-assembly of the “core-shell” micellar structure for drug loading. To quantitate the mitochondria-associated dyes, the mitochondria were isolated from the FITC-material-or Rh123-treated cells. Though the multistep isolation process might reduce the amount of mitochondria-bound dyes, the FITC-F7 showed a doubled mitochondrial accumulation (20.9%) compared with the FITC-PE (11.1%) and its targeting effect was even better than that of Rh123 (17.5%). The mitochondrial membrane potential is an important indicator of mitochondrial health and also a major driving force of lipophilic cation-mediated mitochondrial targeting. In the present disclosure, the mitochondrial membrane potential was measured by the potential-sensitive JC-1 dye that forms red aggregates in healthy mitochondria and becomes green monomers in the cytoplasm when the mitochondria undergo depolarization. The charge-neutral PEG2k-F7 did not significantly change the mitochondrial membrane potential compared to untreated cells, while the lipophilic cation TPP and mitochondrial uncoupler, CCCP, dramatically depolarized mitochondria at the tested dose (FIG. 6B). It was also found that the CCCP-induced membrane depolarization substantially reduced the mitochondrial accumulation of the cationic dye (R123). However, it could not decrease the mitochondrial accumulation of FITC-F7, suggesting that the mitochondrial targetability of F7 and PEG2k-F7 was potential-independent.

To study the mitochondrial binding affinity, the mitochondria were freshly isolated from the untreated cells. Though being electrically neutral, both FITC-PEG2k-F7 and FITC-F7 had strong binding affinities with the isolated mitochondria, which were comparable to those of the potential-dependent (Rh123) and potential-independent (MITOVIEW™ Green) dyes. In contrast, the free FITC or FITC-PEG2k did not significantly bind with the isolated mitochondria. The FITC-PE had a mild mitochondrial binding affinity, in consistent with its nature of the nonspecific intracellular localization. In the competitive binding assay, the TPP pre-incubation decreased the binding affinity of the cationic dye (Rh123) by about 50%, indicating that these lipophilic cations may share the similar binding mechanisms and the membrane potential is a major driving force for their mitochondrial binding. In contrast, the PEG2k-F7 pre-incubation did not influence the binding of Rh123 with mitochondria, confirming that PEG2k-F7 bound with mitochondria in a potential-independent manner. Here, most likely, the hydrophobicity of the fluorocarbon (F7) moiety is the mere driving force of these fluorinated materials' mitochondrial targeting. Without being bound by theory, it is speculated that, in their applications, these charge-neutral fluorinated materials would not alter the mitochondrial membrane potential (health) and their mitochondrial binding affinity would not be influenced by the mitochondrial membrane potential. These features are extremely important for successful mitochondrial imaging and therapy, particularly during cell apoptosis and drug treatment.

Due to the core-shell structure, the PEG2k-F7 micelles were able to carry various hydrophobic/lipophilic drugs for drug delivery. Here, the vitamin E succinate (VES), a widely used “mitocan”, was used as a model drug to evaluate the drug delivery capability of the micelles. Although the PEG-F7 polymers were predominantly accumulated in mitochondria, they did not stimulate the mitochondria to produce reactive oxygen species (ROS) and did not cause obvious cytotoxicity even after 48 h incubation, in agreement with safety studies on the fluorinated biomaterials. In contrast, both the ROS production and cytotoxicity of VES were significantly enhanced when it was loaded to the PEG2k-F7 micelles (PEG2k-F7/VES), compared to those of free VES, physical mixture (PEG2k-F7+VES), and PEG2k-PE/VES micelles, indicating that the increased cellular uptake, endosomal escape, and mitochondrial specificity of the PEG2k-F7 micelles improved the efficacy of the loaded VES. Furthermore, the PEG2k-F7 micelles carrying Rh-PE could efficiently penetrate through the 3D tumor cell spheroids. High cellular penetration and uptake substantially improved the anticancer activity of the loaded VES in the spheroids, as evidenced by the enhanced cytotoxicity and substantial spheroid growth inhibition. Taken all together, the superior performance of the fluorinated materials was most likely connected with the excellent physiochemical properties of the fluorinated micelles as well as the perfluoroalkyl chain's strong capability of binding with cell membrane (cellular uptake), fusing with lipid bilayers (endosomal escape), and interacting with mitochondria (mitochondrial targeting).

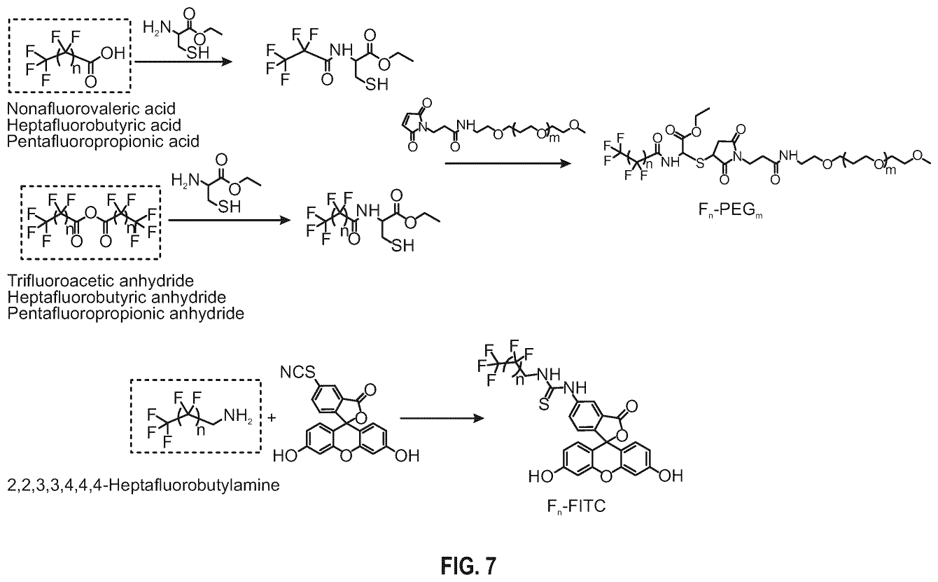

Fluorine-Containing Reagents. FIG. 7 illustrates the synthesis of fluorine-containing reagents according to some embodiments of the present disclosure.

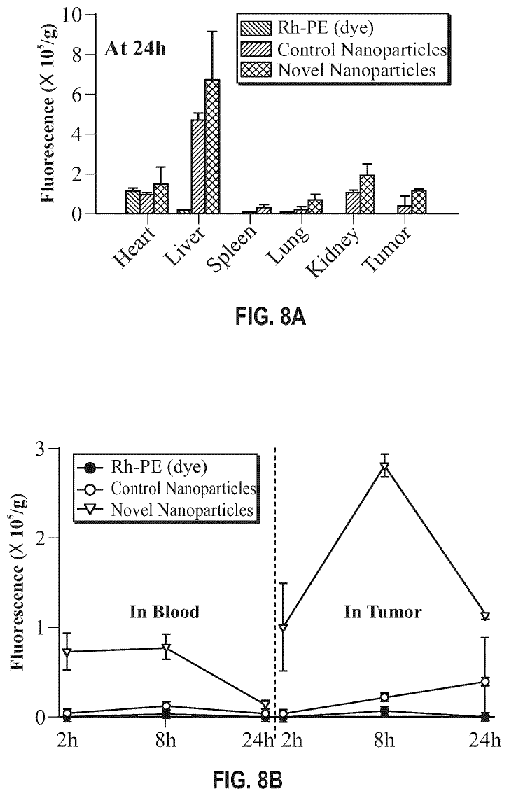

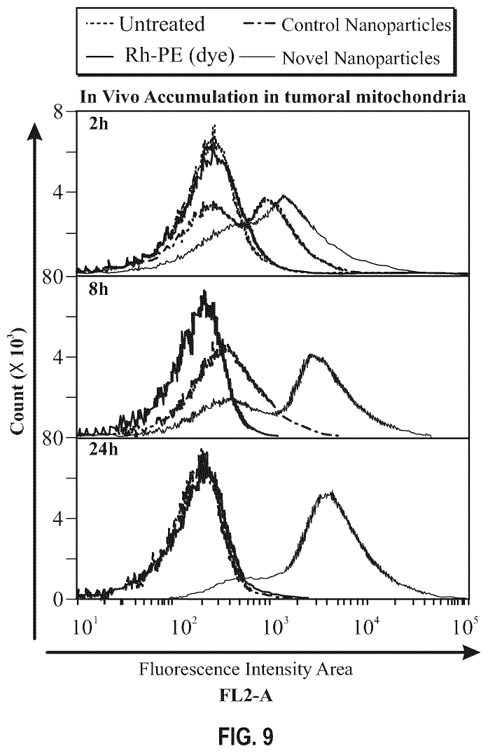

In Vivo Data (i.v. in 4T1 Breast Cancer-Bearing Mice). In vivo, the nanoparticles of the present disclosure demonstrated high accumulation in major organs, tumors, and blood as well as high and persistent mitochondrial accumulation in tumor cells compared to control nanoparticles. For example, FIG. 8A and FIG. 8B illustrates biodistribution of Rh-PE (dye), control nanoparticles, and nanoparticles of the present disclosure in 4Tl breast cancer-bearing mice indicating high and persistent mitochondrial accumulation of the inventive nanoparticles in major organs, tumors, and blood of the present disclosure compared to control nanoparticles. FIG. 9 illustrates in vivo accumulation in tumoral mitochondria for untreated, Rh-PE (dye), control nanoparticles, and nanoparticles of the present disclosure cells over 2 h, 8 h, and 24 h. The nanoparticles of the present disclosure are accumulated in greater concentrations over a longer period of time (24 h) compared to the control nanoparticles.

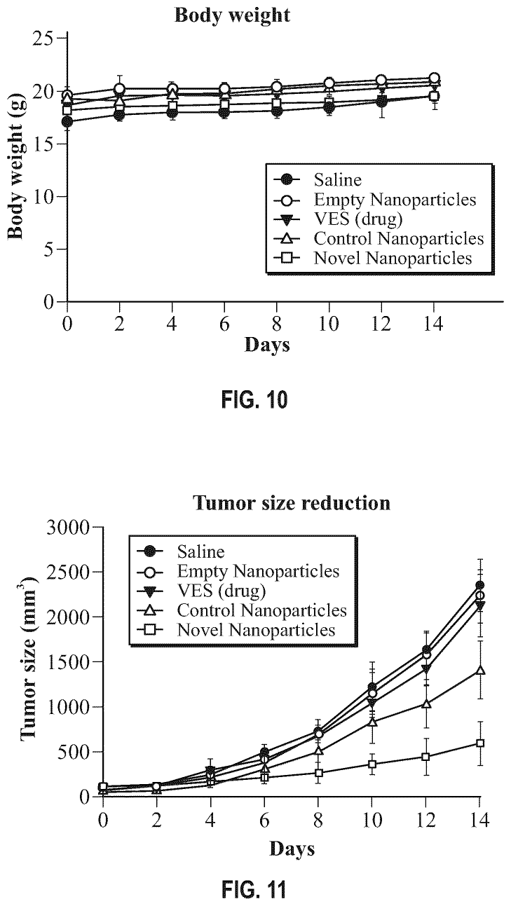

FIG. 10 illustrates body weight of mice treated with saline, empty nanoparticles of the present disclosure, VES (drug), control nanoparticles, and nanoparticles of the present disclosure over 14 days. The graph indicates that the body weight of the mice stayed relatively the same irrespective of whether they were treated with the nanoparticles of the present disclosure, saline, VES or control nanoparticles.

FIG. 11 illustrates the tumor size reduction in mice treated with saline, empty nanoparticles of the present disclosure, VES (drug), control nanoparticles, and nanoparticles of the present disclosure over 14 days. As the graph indicates, the tumors in the mice treated with the nanoparticles of the present disclosure were smaller in size relative to the tumors in the mice treated with control nanoparticles or the anti-cancer drug, VES.

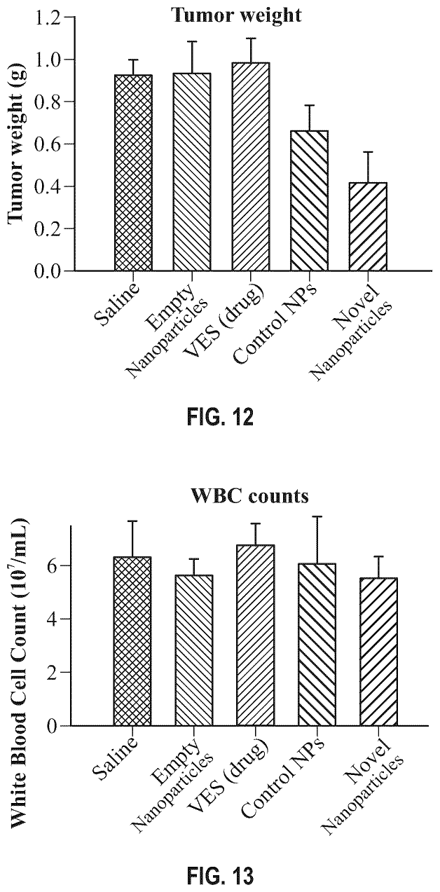

FIG. 12 illustrates tumor weight of mice treated with saline, empty nanoparticles of the present disclosure, VES (drug), control nanoparticles, and nanoparticles of the present disclosure. The graph indicates that the treatment with the nanoparticles of the present disclosure resulted in tumors having a lower weight compared to tumors in mice treated with saline, control nanoparticles or the anti-cancer drug, VES.

FIG. 13 illustrates that WBC counts of mice treated with saline, empty nanoparticles of the present disclosure, VES (drug), control nanoparticles, and nanoparticles of the present disclosure are relatively similar to one another.

As discussed in detail above, the present disclosure demonstrates a reliable intracellular delivery and mitochondrial strategy using the self-assembling fluorinated amphiphilic copolymers as a nanocarrier. Despite being electrically neutral, the PEG-F7 assembled micelles were efficiently internalized by cells, rapidly escaped from endosomes, and preferentially accumulated in mitochondria. The PEG-F7 mediated mitochondrial targeting was potential independent and could deliver various cargoes to mitochondria without compromising mitochondrial health and cell viability. As disclosed herein, the fluorination is a simple and effective way to prepare or engineer functional nanomaterials for targeting of therapeutic and imaging agents to mitochondria.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A fluorinated poly(ethylene glycol) polymer conjugate having the formula Fn-PEGm, wherein n represents the number of F atoms in the Fn fluorine moiety and m represents the molecular weight of the PEG polymer.

2. The conjugate of claim 1, wherein the poly(ethylene glycol) polymer has a molecular weight of 1,000 Da to 5,000 Da.

3. The conjugate of claim 1, wherein n=3-9.

4. The conjugate of claim 1, wherein the poly(ethylene glycol) polymer is covalently linked to the Fn fluorine moiety through a linker.

5. The conjugate of claim 4, wherein the linker comprises an amide bond, an ester bond, a disulfide bond, a thioether bond, or a combination thereof.

6. A micelle comprising at least one fluorinated poly(ethylene glycol) polymer conjugate having the formula Fn-PEGm, wherein n represents the number of F atoms in the Fn fluorine moiety and m represents the molecular weight of the PEG polymer.

7. The micelle of claim 6, wherein the micelle has a mean particle diameter of from about 10 nm to about 500 nm.

8. The micelle of claim 6, wherein the micelle has a mean particle diameter of from about 20 nm to about 200 nm.

9. The micelle of claim 6 further comprising an agent selected from the group consisting of therapeutic agent, a drug, a mitochondria-targeted agent, an imaging probe, a dye or combinations thereof.

10. The micelle of claim 9, wherein the agent is hydrophobic.

11. The micelle of claim 6, wherein the polymer is electrically charge-neutral.

12. The micelle of claim 6, wherein the micelle has a core-shell structure having a hydrophobic core and a hydrophilic shell.

13. The micelle of claim 6, wherein the micelle has a zeta potential between −5 and +5 mV in aqueous environments.

14. A pharmaceutical composition comprising: (a) a plurality of micelles, each micelle comprising at least one fluorinated poly(ethylene glycol) polymer conjugate having the formula Fn-PEGm, wherein n represents the number of F atoms in the Fn fluorine moiety and m represents the molecular weight of the PEG polymer; (b) a therapeutic agent encapsulated within the micelles; and (c) a pharmaceutically acceptable carrier.

15. The pharmaceutical composition of claim 14, wherein the therapeutic agent is hydrophobic.

16. The pharmaceutical composition of claim 14, wherein the plurality of micelles are electrically charge-neutral.

17. The pharmaceutical composition of claim 14, wherein each micelle of the plurality of micelles has a core-shell structure having a hydrophobic core and a hydrophilic shell.

18. The pharmaceutical composition of claim 14, wherein each micelle of the plurality of micelles has a zeta potential between −5 and +5 m V in aqueous environments.
